(19) United States
(12) Patent Application Publication
(10) Pub. No.: US 2018/0286509 A1
(43) Pub. Date: Oct. 4, 2018

(54) Computer-Implemented Multi-Factor Patient Education Method

(71) Applicant: PeerWell, Inc., San Francisco, CA (US)

(72) Inventors: Manish N. Shah, San Francisco, CA (US); Navin Gupta, San Francisco, CA (US); Evan Minamoto, Emeryville, CA (US); Matthijn Dijkstra, Amsterdam (NL)

(21) Appl. No.: 15/927,435

(22) Filed: Mar. 21, 2018

Related U.S. Application Data
(60) Provisional application No. 62/476,256, filed on Mar. 24, 2017.

(51) Int. Cl. G16H 20/40; G16H 10/60; G16H 40/20; G16H 20/60; G16H 20/30; G16H 50/70; G06Q 10/10; G09B 5/06

(52) U.S. Cl. CPC G16H 20/40; G16H 10/60; G16H 40/20; G16H 20/60; G09B 19/0092; G16H 50/70; G06Q 10/1095; G09B 5/065; G16H 20/30

(57) ABSTRACT

A system and method to provide patient education prior to surgery or post-surgery is disclosed. The patient education can be customized based on different factors and provided in a progressive sequence. Patients may be required to complete patient educational lessons and also provide feedback to assess how they are doing. Due to a variety of patient circumstances and the potential for abrupt changes in patient health status prior to, during, or following a health procedure, it is necessary for patient education to adapt to a patient's changing needs. The disclosure describes a technique to assign patient education materials in an adaptive and responsive manner.

### CROSS REFERENCE TO RELATED APPLICATION

This application claims the benefit under 35 U.S.C. § 119(e) of U.S. Patent Application No. 62/476,256, entitled “Computer-Implemented Multi-Factor Patient Education Method” filed Mar. 24, 2017, the entire contents of which is incorporated herein by reference.

### BACKGROUND OF THE INVENTION

The present disclosure is in the technical field of patient instruction. More particularly, the present disclosure is in the technical field of computer-implemented patient education prior to or following any medical procedure. Prehabilitation is often described as physical and/or lifestyle preparation designed to improve recovery time following surgery, although more generally prehabilitation can also improve surgery outcomes and reduce post-surgery complications. Rehabilitation is generally related to the recovery of health through training or therapy after surgery, although more generally pain management is also an important aspect of rehabilitation.

Patients, recommended to undergo a medical procedure by their doctor or a specialist referred to by another doctor, are often not properly educated about the details of the medical procedure, the actions the patient can take to improve the procedure's efficacy (prehabilitation), or how to manage follow-up care after the medical procedure is performed (which is related to rehabilitation).

Currently locations where medical procedures are performed provide patients with education materials in the form of printed brochures and optional classes hosted at the location of the medical procedure. All printed materials are printed for all patients and not customized based on the patient's state of health or current physical condition. Due to the lack of information that is specific to the patient's current health state, patients are forced to visit medical facilities in person or call to ask questions particular to his or her case.

Furthermore, the education materials are not responsive to the changing state of the health of the patient. If the patient begins to experience a health issue (e.g., high blood pressure, fluctuating blood sugar levels, etc.) that could affect the efficacy of the upcoming medical procedure or the patient's ability to recover in a timely manner, the only option available is to delay or cancel altogether the medical procedure. This causes undo physical harm to the patient, lost time of the specialist who would have performed the medical procedure, and extra costs to the facility where the medical procedure would have been performed.

As a response to the lack of patient education that is responsive to the health state of the patient prior to a medical procedure, some health institutions employ extra personnel to communicate with patients before and after a medical procedure. The personnel are typically nurses, case managers, doctors, and specialists and attempt communication with the patient at set times via in-person appointments, phone calls, and other forms of electronic communication. These methods increase the costs borne by the health institution which may not have the funds to employ the right number of people. Furthermore, because of increasing costs, the set times to communicate with patients are not frequent enough to address on-going health state issues of the patient. Additionally, patients may miss the opportunity to speak with the personnel making the communication attempt if the patient is unavailable at the time (e.g., the patient is at work, does not have transportation for an in-person appointment, or forgets the appointment after it has been scheduled).

Other forms of electronic patient education tools do exist to address the issues of accessibility and convenience for the patient. These tools share patient education materials via smartphones, websites, and other computerized applications. However, these software tools do not incorporate patient-specific health information in an adaptive manner to address on-going health issues of the patient as they occur. For example, applications provide a fixed and inflexible amount of patient education material to the patient irrespective of when the patient is scheduled for a medical procedure. If the patient is scheduled to have a medical procedure in six weeks, education materials are delivered to the patient at once, not in a daily cadence. This limits the effectiveness of the education material as patients do not read all the material given and forget to return to material as the procedure date approaches. These tools lack the ability to provide this level of precision and adaptability because it requires a data processing capability that must be scalable to perform computations over hundreds of thousands of variables across a population of people that are set to receive patient education materials. The system designed to perform this processing must also have the storage capacity to surface this information at the precise moment it is needed. Furthermore, these tools do not allow for incorporating new sources of information in the selection of which patient education materials are relevant to the patient at any given time. As such, these tools lack the necessary precision and adaptability to fully serve the needs of any individual patient and their co-occurring health issues. Without incorporating patient-specific health information at the speed at which they can change or adapt education materials is significantly hindered. For example, even if it is known that the patient has a pre-existing condition such as diabetes, these tools do not take that information into account and tailor the patient education material to that specific patient need which might help the patient change their diet to control blood sugar before the medical procedure. Another example might be that the patient is a smoker and should therefore be given more explicit education on the risks associated with smoking prior to a medical procedure such as surgery.

The foregoing examples of the related art and limitations related therewith are intended to be illustrative and not exclusive. Other limitations of the related art will become apparent to those of skill in the art upon reading of the specification and a study of the drawings.

### SUMMARY OF THE INVENTION

The following embodiments and aspects thereof are described in the following description and illustrated in the drawings as systems, tools, and methods that are meant to be examples and illustrative, not limiting in scope. In various embodiments, one or more of the above-described problems have been reduced or eliminated, while other embodiments are directed to other improvements.

The present disclosure describes a computational multi-factor patient education system for providing a series of patient education materials which are responsive to individual patient circumstance and can be adapted based on new information acquired by the patient or from relevant external sources. Patient education materials may be delivered to the patient in an increasingly precise manner.

In one embodiment, a controller selects a sequence of patient education modules for a patient based on different factors. The patient education modules may be provided to a client device of a user in a progression spread out over time. The patient compliance, in terms of viewing the educational content and providing requested feedback or sensor data, may be monitored. In some embodiments, scoring is also performed. Databases of historical data may be created for multiple patients to provide a data set to optimize the selection of educational content and determine the most relevant combinations of factors in terms of patient outcome.

In one embodiment, feedback is collected from each patient as to the accuracy and overall value attributed to each education material visualized to the patient. Here, multi-factor is defined as a system that includes an unlimited number of descriptive attributes about a patient which are used by the system to determine a specific set of patient education materials to assign to a patient at any time. Some descriptive attributes are: patient's known surgery date, patient's diabetic condition, patient's age, patient's gender. Additionally, sensor data from a client device of a patient may also be used to collect data on how a patient is applying instructions and collect information on biomarkers.

In one embodiment, these techniques allow for patients to reach focused, personalized education to prepare them for an upcoming medical procedure or for a medical procedure that has been completed. Patients are able to comply with their doctor's instructions and preferences in order to improve the chances of a successful procedure and a timely recovery. Healthcare stakeholders (e.g., hospital facility, doctors, nurses, etc.) benefit with lower costs and a reduction in time spent with patients prior to or following a medical procedure. Their time is then able to be allocated to other patients that have more strenuous needs and more direct medical attention.

### BRIEF DESCRIPTION OF THE DRAWINGS

FIG. 1A depicts an example of a system for computational multi-factor patient education and support services in accordance with an embodiment;

FIG. 1B illustrates in more detail an example of a system for computational multi-factor patient education in accordance with an embodiment;

FIG. 2 depicts an example of a system for determining and retrieving an assignment of patient education materials in accordance with an embodiment;

FIG. 3 depicts a flow-chart of an example method for multi-factor scoring of a patient education module in accordance with an embodiment;

FIG. 4 depicts another example method for computational scoring function described in FIG. 3 in accordance with an embodiment;

FIG. 5 illustrates a mockup of a smart phone displaying a portion of a patient education module in accordance with an embodiment;

FIG. 6 illustrates a mockup of a smart phone displaying a portion of a physical exercise test in accordance with an embodiment;

FIG. 7 illustrates a mockup of a smart phone displaying a pain management exercise in accordance with an embodiment;

FIG. 8 illustrates a mockup of a smart phone displaying instructions to schedule transportation;

FIG. 9 illustrates a mockup of a smart phone displaying a chat interface;

FIGS. 10A-10D illustrate screen shots of an analytics interface;

FIG. 11 is a flowchart of a method of providing and monitoring patient education in accordance with an embodiment;

FIG. 12 is a flow chart illustrating adapting patient education based on patient response in accordance with an embodiment; and

FIG. 13 is a flow chart illustrating a method of using historical databases of patient medical records, patient education assignment, and patient compliance to make predictions.

### DETAILED DESCRIPTION OF THE INVENTION

High Level System and Support Services Example

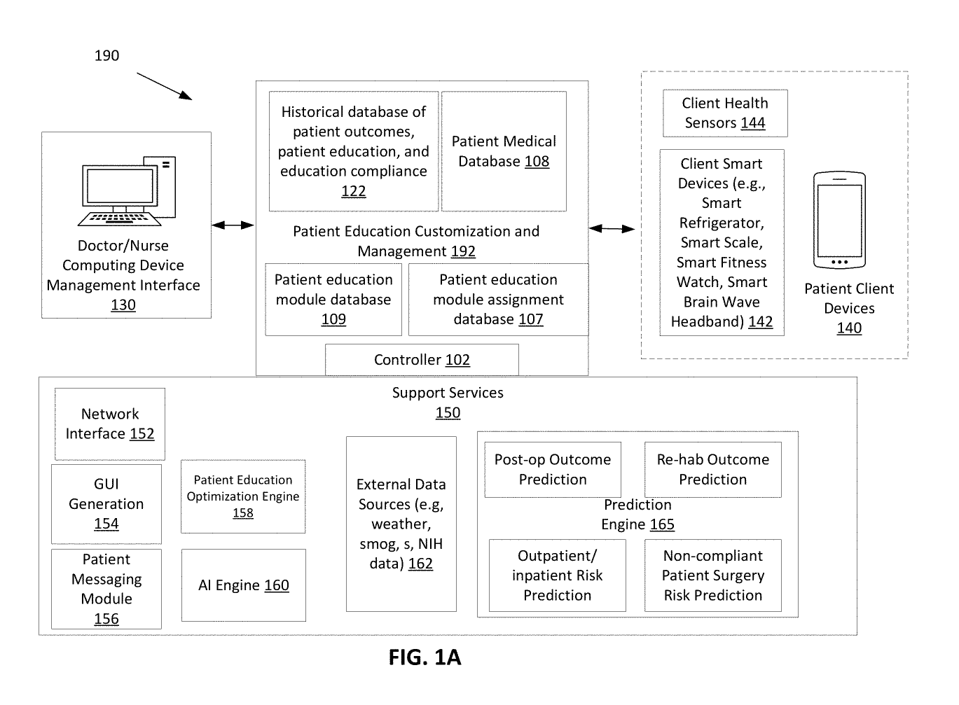

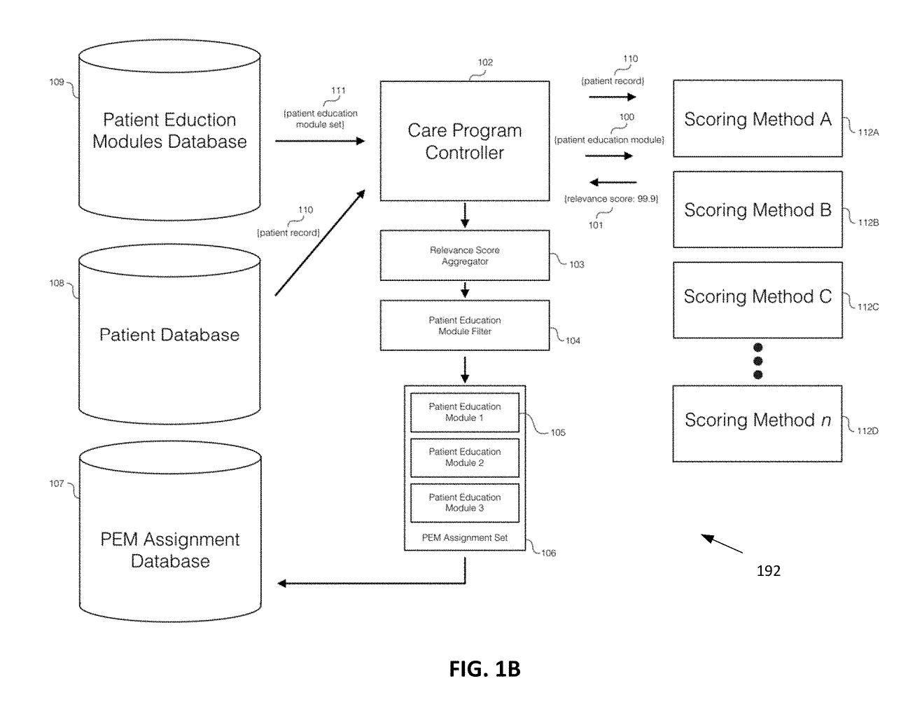

FIG. 1A is a high-level block diagram of an embodiment illustrating a patient education customization and management system 192 in the context of a larger suite 190 of support services 150 and other features. The patient education customization and management system 190 includes a care program controller 102, a patient medical database 108, a patient education module assignment database 107, and a patient education module database 109. The databases may also be mined to generate a historical database 122 of patient outcomes, patient education, and patient education compliance.

The care program controller 102 may be implemented in hardware, firmware, or software and include a processor and memory. The databases may be implemented as a computer storage medium.

One aspect of embodiments of this disclosure is that the patient education modules may be assigned in a progressive sequence based on multiple factors. For example, if a patient has a surgery scheduled in six weeks they could be given a number of lessons spread out over time prior to surgery. For example, they could be provided a short daily education lesson each day leading up to the surgery as part of their prehabilitation. Similarly, after a surgery, a patient may be given a sequence of lessons spread out over time, such as a daily education lesson as part of their rehabilitation.

The selection of patient education modules assigned to a patient may be customized for a particular type of surgery. Some individual portions of the patient education modules may be applicable to educating patients for more than one type of surgery, such as pain management training. Other individual portions of the patient education modules may be specific to a particular type of surgery. Additionally, the sequence of patient education may be customized based on the patient's surgery, their medical history and demographic information, and other factors such as their compliance with earlier patient education modules and feedback from the patient such as survey and sensor data. A partial list of some types of surgery that may benefit include joint replacements, abdominal surgery, organ transplants, bariatric surgery, invasive cardiac surgery, lumpectomy, mastectomy, plastic surgery, and spine surgery. However, it will be understood that this list is non-limiting.

The progression of education that is selected for a patient may be specific to a particular type of surgery, the medical history of the patient, the past or current history of the patient's compliance with education training, or other factors such as the number of days before or after surgery, external data 162 (e.g. weather, smog, etc.), survey data from the patient, or sensor data from a patient's client device 140, smart devices 142, or other client health sensor 144.

In one embodiment, the system is based on a client-server web architecture in which a graphical user interface (GUI) is generated on a patient's client device for the patient to receive education materials and perform interactive exercises. Similarly, a client-server architecture may also be used to provide web access to reporting dashboards to medical doctors, nurses, or insurance companies, depending on implementation.

In one embodiment, the patient's compliance with the education training is monitored. One aspect is that a doctor, nurse, administrator, or other authorized party (e.g., an insurance company) may monitor patient compliance using computing device 130, such as via a computing device having an input, display, and network interface. That is, the management and compliance with education training may be automatically managed and reports generated to verify that patients have received prehabilitation or rehabilitation education. The reports may also indicate whether patients have completed surveys, clicked through education materials, performed interactive education exercises, or otherwise complied with education instructions.

In one embodiment, doctors or nurses may also receive information on a patient's compliance with a set of patient education modules via a client device of a nurse or doctor, such as via a personal computer. For example, a patient education management report module may be designed to issue reports to medical staff of an analysis of the compliance of a patient with a set of patient education modules at scheduled intervals, on demand, prior to surgery, etc.

Another aspect is that patients may receive patient education modules on their client devices 140, such as on smartphones, tablet devices, or home personal computers. Smartphones and tablet devices typically include cameras, microphones, gyroscopes, accelerometers, and sometimes other sensors as well. Also, a patient may have a variety of home smart devices 142, such as a smart fitness watch, a smart brain wave headband, a smart refrigerator, a smart scale, a smart heart rate monitor, etc. Such smart home devices may include a wireless interface to interface with a smartphone or tablet device, thus making it practical for patients to provide additional data inputs to the patient education customization and management system 192.

For example, smartphones include a microphone and a camera. The microphone on the smartphone may be used to measure the strength of a person's breath to determine lung capacity. Smartphones also typically include a gyroscope or an accelerometer. These may be used to observe a patient as they do various tests, such as a sit/stand test, a walking test, or a range of motion test. The camera on a smartphone may be used by patients to take pictures of portions of their body before surgery or after surgery. This may include, pictures of portions of the body pre-surgery or post-surgery for attributes such as discoloration, swelling, and healing. It may also include pictures of wounds post-surgery. Patients may also take pictures of food they eat for analysis via a smart phone's camera. Some applications also permit the sensors in a smartphone to be used to monitor sleep patterns.

Additionally, a variety of other client-side sensors could be employed such as those in smart devices, such as smart scales, smart refrigerators, and smart health watches, or heart rate monitors. For example, a smart scale can report a patient's weight and in some cases their percent body fat. A smart refrigerator can provide an indication of food purchases and consumed. A smart health watch can provide information on heart rate and heart rate variability. Other individual client health sensors also be used. There are also some headbands that monitor brainwaves that could be used, in combination with other information, to determine if a patient is following a suggestion to engage in a guided meditation

In one embodiment, the support services 150 include a network interface 152 (e.g., an internet interface), a graphical user interface 154, a patient messaging module 156, and a patient education optimization engine 158. In some embodiments, an artificial intelligence (AI) engine 160 is provided to analyze the historical database 122, optimize the selection of patient education modules, notifications, and reminders. Interfaces may be provided to access external data sources 162 such as weather, smog, etc.

A prediction engine 165 may be provided to analyze the historical database 122 and make relative statistical predictions about patient outcomes with respect to the historical database. For example, a patient's medical history and their compliance with education modules may be used to generate information on whether they have a greater than average risk of post-operation problems or complications. For example, a patient that does not comply with education instructions for exercise and diet may have a statistically higher risk of post-surgery complications. As another example, a prediction may be made of whether a patient is a reasonable risk for an inpatient procedure. For example, patients who are very diligent in complying education instructions may be more likely than average to follow education instructions after an inpatient procedure. Additionally, predictions may be made regarding rehabilitation outcomes. For example, a patient who completes a pain management course may be more likely to be able to handle pain (with minimal medication) in the weeks after surgery. In some embodiments, the prediction engine may also be implemented based on training the AI engine 160 using data form the historical database 122.

Additionally, feedback may be solicited from patients on the subjective value they receive from the educational materials and the course of instruction they receive as a whole. For example, patients can be polled on their subjective opinions on the value to them of individual patient education modules or portions thereof. This additional subjective feedback could also be stored in a historical database and used to adapt educational content for a current patient or future patients.

Additional Multi-Factor Use Scenarios and Multi-Factor Compound Biomarkers

One aspect of embodiments of the present disclosure is multiple factors may be used to customize a progression of educational content provided to a patient. Some of the individual factors may include biomarkers of physical health that can be determined utilizing the sensors of the client devices of patients. However, surveys of patients may also provide additional important information. For example, pain surveys of a patient may provide information on the subjective levels of pain a patient experiences on a regular basis or in doing specific activities. Depression surveys are also useful given the connection between depression and patient energy levels and general physical health.

The information collected from patients for each patient education module may also include multiple factors, including in some embodiments compound biomarkers. For example, a compound biomarker may include two or more forms of feedback. In the most general case, a total of N different factors may be considered in combination to form a compound biomarker that can be scored. In some embodiments, the particular score for a compound biomarker can then be used to determine additional education provided to the patient or a sequence of additional biomarker tests in additional patient education modules.

In one embodiment, compound digital biomarkers may be collected from patients based on the combination of different feedback from the patient. For example, the sensors in a smartphone may be used by a patient to generate a measure of knee flexion or knee extension as described below in more detail by the patient placing the smartphone on a portion of their leg so that when a knee flexion or knee extension exercise is performed the smartphone moves as well, with the movement sensors of the smartphone reporting the range of motion.

As another example, the motion sensors on a smartphone may be used to generate an estimate of a step count (number of steps taken).

This data may be combined with information from pain surveys and depression surveys in different permutations to generate a compound biomarker. Some examples include:

1) a smartphone based knee flexion+pain survey (e.g. a pain survey on the VAS scale);

2) a smartphone based knee extension+pain survey (e.g., a pain survey on the VAS scale);

3) a motion sensor step count+pain survey (e.g., on the VAS pain scale);

4) a depression survey (e.g., a depression, anxiety, a stress scale (DASS) such as the DASS21);

5) a depression survey (e.g., patient health questionnaire 9 (PHQ9))+a pain survey (e.g., on the VAS scale)+a step count.

The use of compound biomarkers is not limited to these examples. Generally speaking, combining several different sources of information provides a more comprehensive way to understand the current mental and physical health of a patient in order to customize his education pre-surgery and post-surgery. The compound biomarkers may also be scored and the scores stored for later analysis to understand how individual biomarkers and compound biomarkers changed over time through patient education.

Other examples of functional physical exercise measurements include a sit/stand test. For example, a smartphone's sensors (e.g., gyroscope and accelerometer) may be used to observe a patient as they do a sit/stand test. There are a variety of sit/stand tests. In some of these a patient is asked to quickly move from a sitting to a standing position a set number of times (e.g., 5). The sit/stand test can be used to determine patient strength, balance, and coordination for the patient pre-surgery and post-surgery. The information can also be used as a good predictor of a likelihood that a patient is at risk of falling.

A smartphone microphone can also be used to measure the strength of a person's breathing to determine lung capacity, similar to a spirometer. This information can be use in determining which physical exercises to give to a patient

As yet another example, a patient could be asked to walk a certain number of steps or walk for a given length of time (e.g., 6 minutes). The smartphone's motion sensor may be used to observed the patient's motion while he walks.

As another example, nutrition is also a factor in patient health. A healthy diet may improve mood and may reduce inflammation. In one embodiment, a patient takes a picture of the food he eats, which may then be analyzed for nutritional content based on the photos. More generally, the patient may also take photos of barcodes of pre-packaged food he consumes. Other information sources, such as data from smart kitchen appliances or receipts from food purchases may be used to infer the patient's diet. The dietary information and its nutritional analysis may be combined as a compound digital biomarker with other data such as patient activity levels, patient range of motion, pain assessments, and depression surveys. In one embodiment the food and nutritional data may be collected and a database generated to determine, for particular medical conditions and surgeries, the impact of diet/nutrition on other markers such as pain, activity, and range of motion for particular medical conditions.

Photographic data may also be collected. This may include pictures that can be analyzed to show discoloration, swelling, skin condition, body sores, tongue, eyes, etc. Post-surgery the pictures may include pictures of post-surgery wounds. This photographic data can be combined with information such as pain assessments, range of motion, and activity levels. For example, in the case of post-surgery, photographs of a wound may provide an indication of wound healing, which in turn may indicate whether a patient is at risk for infection or should change their dressing more often.

Sleep habits may also be monitored and combined with other data in the system. For example, a patient may be given surveys about his sleep habits or his sleep habits may be actively monitored. This sleep habit data can be combined with other data such as activity levels range of motion, pain assessments, and depression surveys. In one embodiment the sleep habit data may be collected and a database generated to determine, for particular medical conditions and surgeries, the impact of sleep habit on other markers such as pain, activity, and range of motion for particular medical conditions.

As another example, heart rate monitoring data may be gathered about a patient's heart rate and heart rate variability. The heart rate and heart rate variability data may be combined with other data to create combined compound biomarker.

Additionally, in some embodiments, a patient may also authorize other personal information to be submitted, such as credit card information or global positioning satellite (GPS) data that might be mined for information relevant to a patient's diet, exercise, or other health practices. For example, GPS data and local weather data might be used to suggest exercise routines consistent with the patient's medical conditions, local weather, local smog, or local pollen conditions. As one example, in some environmental conditions, a patient could be advised to perform indoor exercise. In other environmental conditions, the patient could be advised to take a short walk outside for the psychological benefits of being outside in the fresh air and sunshine for its anti-depression benefits.

In one embodiment, patient education modules 100 that are selected may be customized for preparing a patient for a specific medical procedure. As an example, a somewhat different medical education regime may be utilized for preparing a patient for a joint replacement procedure for a knee than for a hip. A different medical education regime may be utilized for an Anterior cruciate ligament (ACL) reconstruction operation. As still yet another example, a medical education regime to prepare a patient for spine surgery may utilize still yet a different medical education regime.

A variety of different patient data may be collected as part of the patient education modules and/or performing an evaluation of the effectiveness of the education. Patients may be scored in different ways. For example, in one embodiment patients may be scored based on the behavior of their use of a graphical user interface of an application implementing the patient education modules. For example, patients may be scored on how diligently and thoroughly they view education materials and implement assigned tasks. For example, if an education module includes a video training then a patient may be scored based on whether or not he watches the entire video. If the education module includes a user input or requires user feedback, then the patient may be scored on that as well.

In one embodiment patients are given surveys, such as pain surveys. More generally, a variety of assessment surveys may be performed. For example, pain management has many psychological elements. Taking one or more pain surveys or depression screen surveys prior to surgery can be an important source of information in assessing a patient's ability to handle pain and in some cases training them to handle a greater level of physiological pain. Additionally, the pain surveys may be conducted in combination with other tests, such as asking the patient to perform a specific exercise routine and having the patient fill out a pain survey for that particular exercise. For example, for a knee operation a patient may be asked to perform an exercise to measure the range of motion of their knee and provide a score of the pain he experiences during the exercise.

In one embodiment, patients are given depression screens. Patients can become depressed prior to surgery and this can be detrimental in a variety of ways. The depression screens can, for example, include surveys. However other factors could be considered. For example, weather (such as the hours of sunshine per day) can be a contributing factor to depression in some cases dues to seasonal affective disorder. Thus, a patient with an operation scheduled in darker winter months may be more at risk for depression.

In one embodiment, sensor data may be taken of a patient, such as via the sensors of a smart phone. For example, the accelerometers, gyroscopes, and other features of smartphone may be activated when a patient is assigned an exercise task to generate sensor data. For example, patients can be asked to walk for a specified length of time and their motion monitored by the sensors of the smartphone. A range of motion of the patient, such as a range of motion of a knee, may be measured by placing the smartphone on a patient's leg and instructing the patient to flex his leg. Some aspects of the walking gait of a patient may also be analyzed via common smartphone sensors. Other external devices, such as a smartwatch or Fitbit® may be used to generate data on a patient's exercise routine. Heart rate, heart rate variability, and respiration may be measured using common sensors.

Another aspect of preparing for a surgery may include home preparedness. For example, the risk that a patient falls in his home pre-surgery or post-surgery may depend on the patient preparing his home to have a desired clearance for moving around with crutches or a wheelchair, etc. More generally, there may be other tasks a patient may need to do prior to surgery to prepare their home. In one embodiment, a video camera is used by a patient (e.g., a patient's smartphone video camera) to capture images of the patient's home. In one embodiment, this may be used to verify that the patient is taking steps to prepare his home prior to surgery. In another embodiment, video images of the patient's image may be used to perform hazard detection, such as detecting objects or obstacles that may cause the patient to trip. For example, portions of a home not satisfying crutch or wheelchair safety parameters could be identified. Other portions of a home posing a risk for tripping could be identified. In one embodiment, the AI engine is trained to examine images of a patient's home, identify potential risks, and generate recommendations.

In theory other sources of information could be mined as sources of data to assess whether or not a patient is complying with education guidelines in a patient education module. This could, for example, include having the patient report on home health tests (e.g., weight) or making inferences from inputs (e.g., data that may be indicative of a patient following recommended dietary guidelines, such as data that may be inferred from credit card receipts, smart refrigerators, etc.)

It is also possible to adapt the patient education modules based on other sources of data. For example, if there is heavy smog, high pollen counts, or poor weather, an exercise recommendation in a patient education module could be modified and/or its scoring modified. For example, a patient with a knee injury should probably not be walking outside pre-surgery if there is ice and snow that could create a risk of a fall.

In one embodiment, an objective of the care program controller 102 is to perform a multifactor analysis and select a sequence of one or more patient education modules for the patient that is most likely to produce the best outcome or outcomes for the patient. The sequence may also be selected to provide education and interactive exercises that are spread out over time.

A best outcome may be defined in various ways depending on implementation details. For example, the best outcome could be defined in terms of preparing the patient for a successful surgery in terms of likelihood of a successful surgery according to some statistical metric, a reduced risk of post-surgery complications, speed/ease of recovery/rehabilitation, etc. Also, another factor that might be considered in some embodiments are cost considerations such as those of insurance companies.

While it is difficult to quantify outcomes for individual patients, general statistical levels of risk may be determined for large groups of patients facing the same surgery and having comparable demographic and medical data for a given degree of compliance with a patient education module. Thus, at least a degree of prediction of patient outcomes may be determined in a statistical sense in terms of high or low risk relative to some average risk.

For example, a patient who doesn't comply with some specific requirements of patient education modules may be at a greater risk compared to a patient with average compliance facing the same surgery and similar demographic and medical information. For example, a patient who doesn't perform recommended exercises at all may be at a higher risk compared to an “average” patient who performs the exercises but “cheats” a little such as skipping a few days here and there. Conversely, a patient who diligently applies himself to exactly follow all of the instructions of a set of patient education modules may have a lower risk compared to an average patient facing the same surgery and having comparable demographic and medical information.

The compliance of a patient with a set of one or more patient education modules may be scored. The scoring process may be used generally to determine compliance with a recommended set of one or more patient education modules. However, more generally it may be used to collect patient data that may be used in a variety of ways, such as to select additional patient education modules for the patient.

In some embodiments, predictive modeling is used to assess risks. This may include assessments of risks of in-patient vs. out-patient surgery. For example, in some cases, patients who diligently follow pre-surgery education guidelines are better candidates for out-patient surgery for objective physiological reasons such as losing weight (if they need to lose weight) and exercising (if they need to exercise) prior to surgery. Also, a patient's proven ability to precisely follow education instructions may also be useful in assessing his ability to follow instructions after out-patient surgery, thus making him a better candidate for out-patient surgery.

Predictive models may also be used, to some extent, to predict post-surgery recuperation, which may be important for estimating when a patient can return to work. For example, since pain management has a strong psychological component, a patient who has diligently complied with pre-surgery education training regarding pain management may have a better recuperation, in terms of pain management, than a patient who did not comply with pre-surgery education training. Additionally, a patient who diligently complies with pre-surgery education training may also have a better recuperation for physiological reasons, such as a patient following a patient education module recommending losing weight prior to a knee replacement operation, etc.

The benefits to a patient may depend in part on the type of operation he is being prepared for. For example, in some common muscular-skeletal procedures, the benefits to a patient of being provided a customized patient education module may include in some cases shortened stays in a hospital after surgery, a reduced rate of re-admission for post-surgery complications, the possibility in some cases of out-patient surgery, and the possibility in some cases to go immediately home after surgery and perform rehabilitation at home. Doctors also benefit by virtue of a reduction in the management burden to educate patients.

In some embodiments, intelligent reminders or notifications may be sent to patients about things to prepare for, such as lab tests, blood banking, and scheduling rides to/from the hospital. Notifications can also be sent about particular physical therapists located near a patient and/or that are within a patient's insurance plan. The reminders can also include reminders to purchase household items such as grab bars, bath mats, or other items that help prevent falls or make it easier to recover at home. The reminders can also indicate items covered by the patient's insurance plan.

In one embodiment, intelligent reminders or notifications are sent to patients regarding particular health conditions. As one example, if a patient is a diabetic, he can be reminded to keep his blood sugar under control. As another example, if a person is at a high risk for depression or anxiety he can be reminded to engage in a pre-operative pain management and calming exercises, such as meditation and breathing exercises. As another example, if a person is not active enough, the system can remind him to reach specific activity milestones. In some embodiments, the intelligent reminders and notifications can be based on the person's age, gender, surgical procedure, etc.

In one embodiment, the intelligent reminders and notifications may be determined via the AI engine from the historical data collected of patients who go through prehab and rehab. As more historical data is collected, the effect of reminders and notifications can be adapted and customized based on patient demographics, environment, surgical procedure, care setting (inpatient vs outpatient) to optimize the intelligent reminders, notifications, and educational content.

The selection of one or more patient education modules in a set of patient education modules by a care program controller 102 may include basing the selection at least in part on the type of operation the patient will have. For example, the pre-surgery education to prepare for a knee replacement would typically include aspects different than the patient education to prepare for a spine surgery.

The selection of a patient education module by the care program controller 102 may also take into account other aspects of the individual patient's demographic data and medical history, as will be described below in more detail. For example, a patient's pre-surgery weight may be a more important consideration for some surgeries than for others. Having a patient measure knee mobility may be more important for knee operation than spinal surgery and so on. In any case, the care program controller may access the patient database and determine the operation the patient is scheduled to have, the date that it is scheduled for, pertinent medical information about the patient relevant to the operation, and then select a set of at least one patient education module for the patient.

As an illustrative example, the patient education modules 100 may include but are not limited to: strength training exercise descriptions and instructional videos, mobility exercise descriptions and instructional videos, nutritional education specific to blood sugar control with accompanying instructional videos, nutritional education specific to increasing protein and vitamin levels prior to surgery with accompanying instructional videos, pain management exercises with accompanying audio guides, home preparedness education material, interactive exercises to assess range of motion, interactive activities to request a car service for pickup, and others. However, it will be understood that they are merely examples and that additional possibilities are possible.

Additional System Examples

FIG. 1B depicts in more detail an example of system 192 used to retrieve a patient education module set 111 from a patient education modules database 109 and a patient record 110 from a patient database 108 via a care program controller 102. In one embodiment, a patient record 110 is comprised of a unique identifier and health information shared by the patient including weight, height, age, other vital signs, existing health conditions like high blood pressure or diabetes, etc.

The patient education module set 109 is comprised of patient education modules 119. These patient education modules may provide education for a patient pre-surgery. However, more generally they may also provide post-surgery education.

In one embodiment, each patient education module 119 is provided via a graphical user interface on a client computer or smartphone.

In one embodiment the monitoring of patient compliance may include scoring. As previously discussed, a patient may be assigned a progression of patient education modules. For example, every day prior to surgery a patient may be given reminders, instructions, diet and exercise lessons, home preparedness instructions, pain management lessons, etc. A single day's education may include more than one aspect. Thus, a variety of different scoring methods may be applied to a single day of patient education and the cumulative patient education. For example, a first scoring method could score whether a patient has viewed the content of a patient education module by, for example, clicking through one or more page. Another scoring method could be based on when the patient views the materials. Another scoring method could be applied for surveys the patient responds to. Still another scoring method could be applied to any sensor data received from a patient client device. Thus, for a particular portion of an education module, one or more scoring methods may be selected. For example, a single patient education module providing several short pages of content could have a first soring method regarding when it was viewed, a second scoring method regarding how completely it was reviewed, and another scoring method for a simple question and answer test used to help the patient memorize the instructions of the educational module.

In one embodiment, the Care Program Controller 102 iterates through the set of patient education modules 100 and applies at least one scoring method 112A-112N used to score each patient education module 100.

In one embodiment, a relevance score 101 is output by each scoring method 112A and associated with the specific patient education module 100 and the patient record 110. Scoring methods 112A-112N are used at different times at the discretion of the Care Program Controller 102. For example, scoring methods 112A-112N may be used only for patients that have yet to undergo the medical procedure or only for patients that have gone through the medical procedure.

In some embodiments, other scoring methods 112A-112N are used for both before and after medical procedures such as patient education modules 100 that provide nutritional education. For example, in one embodiment, each patient education module 100 gets a set of scores (one score for each scoring method applied to it). The scoring method may determine a different score for each module based on how it is developed.

As an illustrative example, consider a scoring method based on recency. As an example of this, if PEM 1 was assigned and completed within the last 7 days, a Recency Scorer would generate a low score for PEM 1, if PEM 2 has not been assigned before, the RecencyScorer would generate a higher score for PEM 2. The Score Combiner would see the see the score generated by the RecencyScorer and other scorers and determine the overall score for PEM 1.

In some embodiments, the Care Program Controller 102 includes a processor and memory. The processor may execute software instructions by performing various input/output, logical, and/or mathematical operations. The processor may have various computing architectures to process data signals including, for example, a complex instruction set computer (CISC) architecture, a reduced instruction set computer (RISC) architecture, and/or an architecture implementing a combination of instruction sets. The processor may be physical and/or virtual, and may include a single processing unit or a plurality of processing units and/or cores. In some implementations, the processor may be capable of generating and providing electronic display signals (e.g., a visual dashboard) to a display device, receiving and processing continuous stream of data, performing complex tasks including various types of data attribute extraction and query execution, etc.

The memory may be included in a single computing device or distributed among a plurality of computing devices as discussed elsewhere herein. In some implementations, the memory may store instructions and/or data that may be executed by the processor. The instructions and/or data may include code for performing the techniques described herein. The memory is also capable of storing other instructions and data, including, e.g., an operating system, hardware drivers, other software applications, databases, etc. The memory may be coupled to the bus for communication with the processor and the other components of the system. The memory may include one or more non-transitory computer-usable (e.g., readable, writeable) device, a static random access memory (SRAM) device, an embedded memory device, a discrete memory device (e.g., a PROM, FPROM, ROM), a hard disk drive, an optical disk drive (CD, DVD, Blu-ray™, etc.) mediums, which can be any tangible apparatus or device that can contain, store, communicate, or transport instructions, data, computer programs, software, code, routines, etc., for processing by or in connection with the processor. In some implementations, the memory may include one or more of volatile memory and non-volatile memory. For example, the memory may include, but is not limited to, one or more of a dynamic random-access memory (DRAM) device, a static random-access memory (SRAM) device, an embedded memory device, a discrete memory device (e.g., a PROM, FPROM, ROM), a hard disk drive, an optical disk drive (CD, DVD, Blu-ray™, etc.). It should be understood that the memory may be a single device or may include multiple types of devices and configurations.

In one embodiment, the Care Program Controller 102 exhausts all scoring methods 112, it will use the relevance score aggregator 103 to combine relevance scores 101 for a patient education module 100 and patient record 110 into a final score 301 representing the final value for patient education module 100 and patient record 110.

In this instance, the pairing of a patient education module 100 and a patient record 110 during this computation flow is a unique pairing to the system. The unique pair is maintained globally once the final score 301 is determined. Upon receiving all scores for a patient education module 100, the relevance score aggregator 103 combines the scores based on internal weights and preferences associated with each Scoring Function 112. The final aggregation is done by the relevance score aggregator 103 through a set of methods such as simple sum of all relevance scores 101. Another aggregation could be the weighted sum of the relevance scores 101.

In one embodiment, the Care Program Controller 102 then uses the patient education module filter 104 to reduce the patient education module set 101. Patient education modules 100 are removed from the set based on an internal schema for determining the minimum relevance score thresholds for any patient education module 100. For example, in one embodiment, the Care Program Controller 102 iterates through the set of patient education modules 100 and keeps the ones that fit the internal schema or criteria. Then the Care Program Controller 102 creates a new final collection after applying the internal schedule to each module and selecting the ones that pass Boolean criteria. An example of this schema could be to only include relevance scores 101 above 90.0. Relevance scores 101 in this example are values between 0 and 100 but other numerical and non-numerical scales can be used.

In one embodiment after the patient education module set 111 is filtered, a PEM Assignment Set 106 is created and stored in the PEM Assignment Database 107. For example, in one embodiment, when PEM Assignment Set 106 is saved, the logical record is unique to this computational run of the system. If we run the system again, it may generate a different set based on new information so the records here are unique based on the information available at the time it was created. In one embodiment, the system stores the underlying scores as well so they can later be used to determine why it was chosen by the system.

In one embodiment, a PEM Assignment Set 106 is a logical record that contains the patient education modules 100, the patient record 110, the relevance scores 101 for each patient education module 100, and the date when this assignment was created. The PEM Assignment Set 106 is then stored in the PEM Assignment Database 107 for future use.

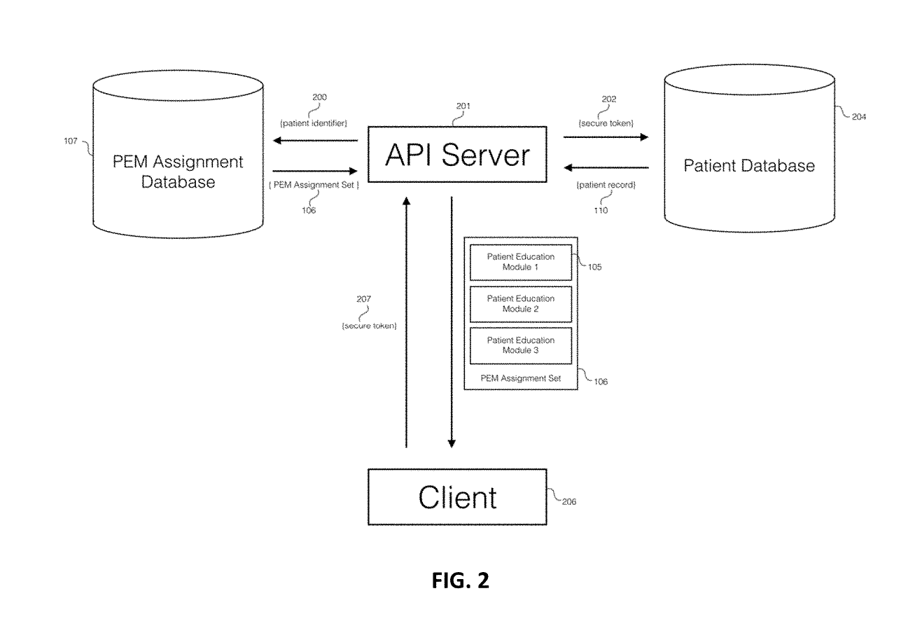

FIG. 2 depicts an example of a system used to retrieve a PEM Assignment Set 106 through a secure API server 201 via a client 206. The client 206, using a secure token 207, requests one or more PEM Assignment Sets 106 from the secure API server 201. The secure API server 201 validates the secure token 207 and then retrieves a patient record 110 associated with the secure token 207. Then the secure API server 201 makes a separate retrieval request to the PEM Assignment Database 107 with a patient record 110 to retrieve a PEM Assignment Set 106. If a valid PEM Assignment Set 106 is found, the secure API server 201 retrieves it and sends it to the client 206 in the response. The client 206 may request one or more PEM Assignment Sets 106 associated with one or more patient records 110. The secure API server 201 will only respond with PEM Assignment Sets 106 which are associated with the patient record 110 and that the client 206 has permissions to receive. Additionally, the client 206 can specify parameters as to which PEM Assignment Sets 106 to retrieve. For example, in some embodiments, the client 206 may only need to retrieve PEM Assignment Sets 106 that were created for the patient record 110 within in a predetermined amount of time, e.g., in the last week.

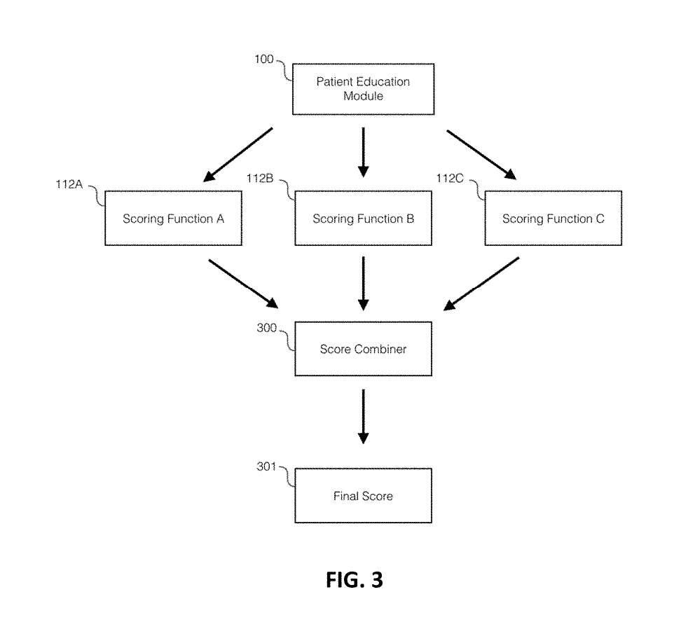

Because of the variety of patient circumstances and the potential for abrupt changes in patient health status, the selection of patient education modules 100 must be able to adapt to patient needs in real-time. FIG. 3 depicts an example of this whereby the Care Program Controller 102 has access to a range of scoring methods 112A-112N. A determination is made in each instance of use of the Care Program Controller 102 as to which scoring methods 112A-112N are applicable for the patient record 110 for whom a new PEM Assignment Set 106 is being created.

For example, a patient may be having his procedure performed in 14 days from the current instance and only scoring methods 112A-112N that are time sensitive need to be utilized. Other Scoring Methods 112A-112N which do not meet this criterion are not considered.

FIG. 3 depicts a workflow for the care program controller 102 in choosing from a set of a scoring methods 112. Scoring methods 112 can be added and removed at any time and therefore the scoring methods 112 shown in the diagram are only for illustrative purposes and do not represent a fixed number of scoring methods 112. For example, in some embodiments, different methods and combinations of weighting, normalizing and filtering maybe applied including simple summing of the scores, weight-adjusted summing, etc.

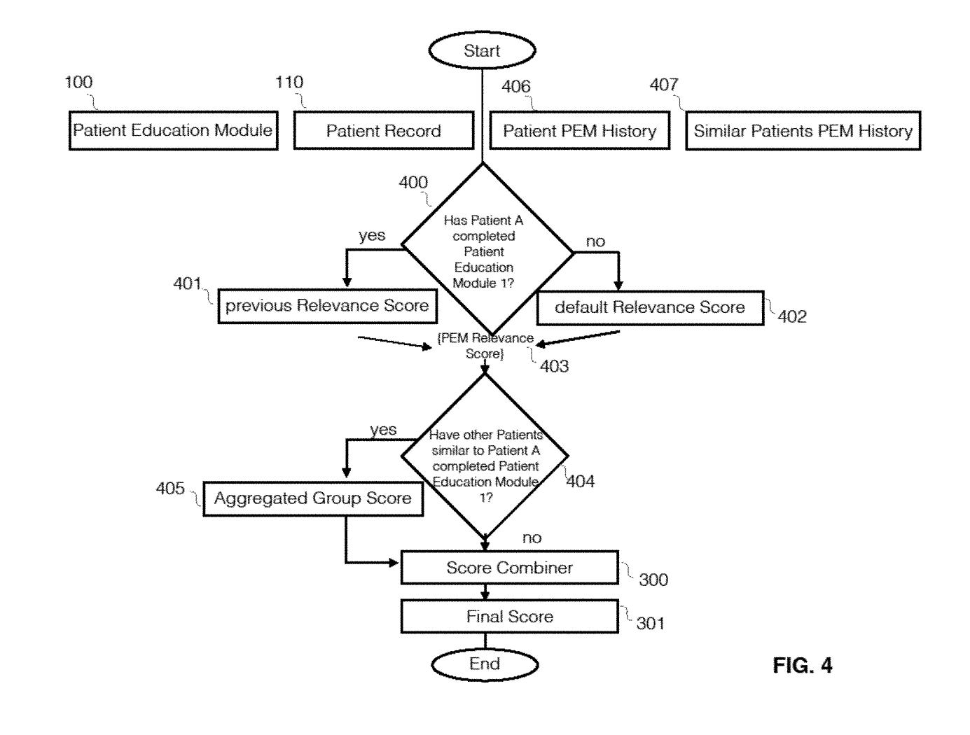

FIG. 4 depicts an example of a workflow that could be used by a scoring method 112. In this example, the scoring method 112 is given as input in a patient education module 100, a patient record 110, records related to the patient education modules 100 that have been previously assigned to the patient (Patient PEM History 406), and records related to similar patients that have been assigned the patient education module 100 not including the active patient (Similar Patients PEM History 407).

In one embodiment, the first step determines if the patient has previously been assigned the given patient education module 100. If yes, the relevance score 101 from the previous assignment is used as the PEM Relevance Score 403. If no, a default relevance score 101 is used as the PEM Relevance Score 403. In one embodiment, the second step determines if other patients have been assigned the given patient education module 100 previously. If yes, the relevance scores from the previous assignments are aggregated and output as a single Aggregate Group Score 405. An example of an aggregation could be the average of all the relevance scores 101, or selected ones or groups of the relevance scores. Other criteria, e.g., determined by the data, may be used to select particular relevance score for aggregation including doctor preference, previous patient outcomes, etc.

In one embodiment, the scoring method 112 then combines the two relevance scores (the Aggregate Group Relevance Score 405 and the PEM Relevance Score 403) using a score combiner 300. An example of a score combiner 300 could be the weighted average of the PEM Relevance Score 403 and the Aggregate Group Relevance Score 405. After combining into a single final score 301 the scoring method returns the value as its relevance score 101.

User Interface Examples

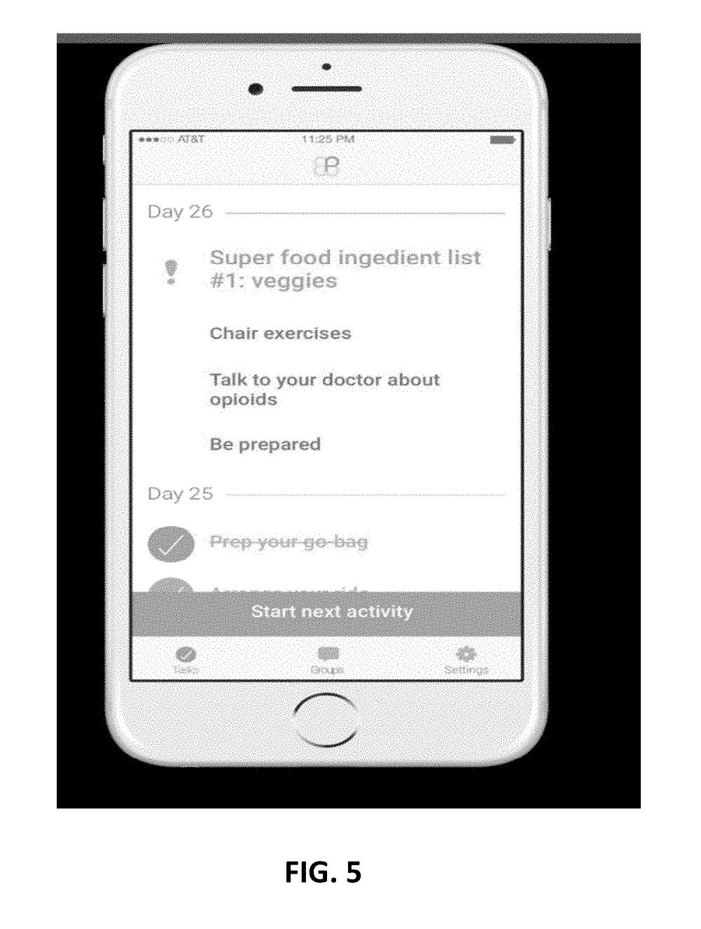

FIGS. 5-10 illustrate a few examples of user interfaces, although it will be understood that this disclosure is not limited to a particular user interface design.

FIG. 5 is a mockup of a smartphone displaying a patient education module. In this example, an instruction is provided for a particular day (e.g., day 25). The patient is given a list of several different educational task activities. In one embodiment the patient taps/clicks through individual task items to access more information or to engage in interactive exercises. For example, a dietary recommendation to eat more vegetables could be tapped to access additional dietary information. A chair exercises section could be tapped to open up that day's chair exercises. And so on, throughout a sequence of educational materials and reminders. In this example, the patient is given a specific set of tasks for each day in a sequence with respect to a particular surgery.

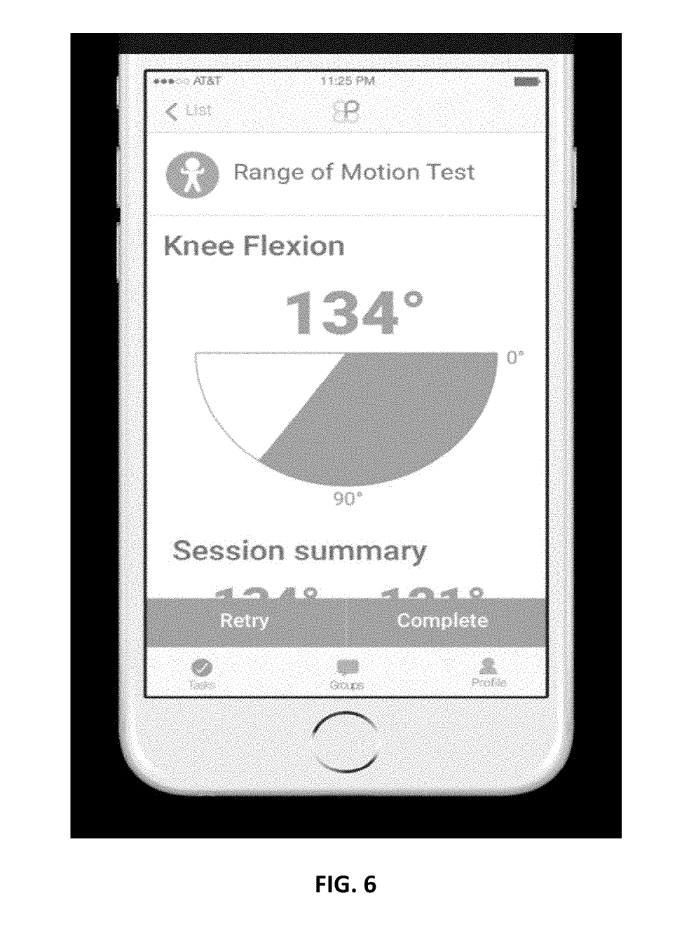

FIG. 6 illustrates an example of a smartphone displaying an exercise routine. In this example, a knee flexion test is provided. In one embodiment, a patient puts his smartphone on a portion of his leg below the knee and flexes his knee. Using the sensors of the smartphone a new flexion angle can be calculated and optionally displayed to the patient. They patient may also be request to record his pain level.

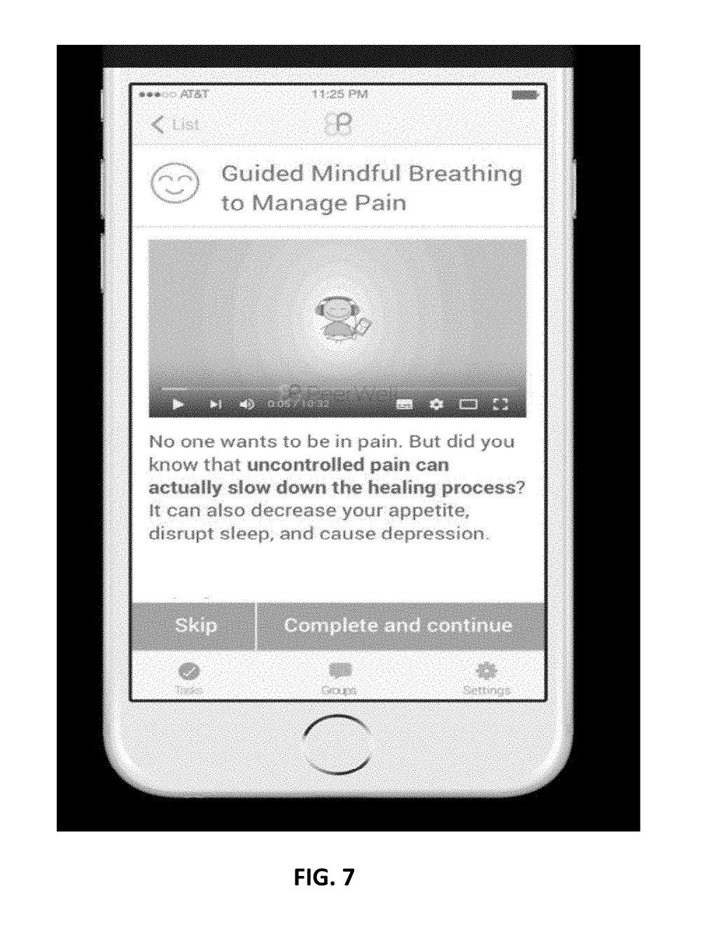

FIG. 7 illustrates an example of a smartphone displaying a guided meditation breathing exercise to manage pain. In this example, a user may be provided with an audio or video providing the meditation instructions.

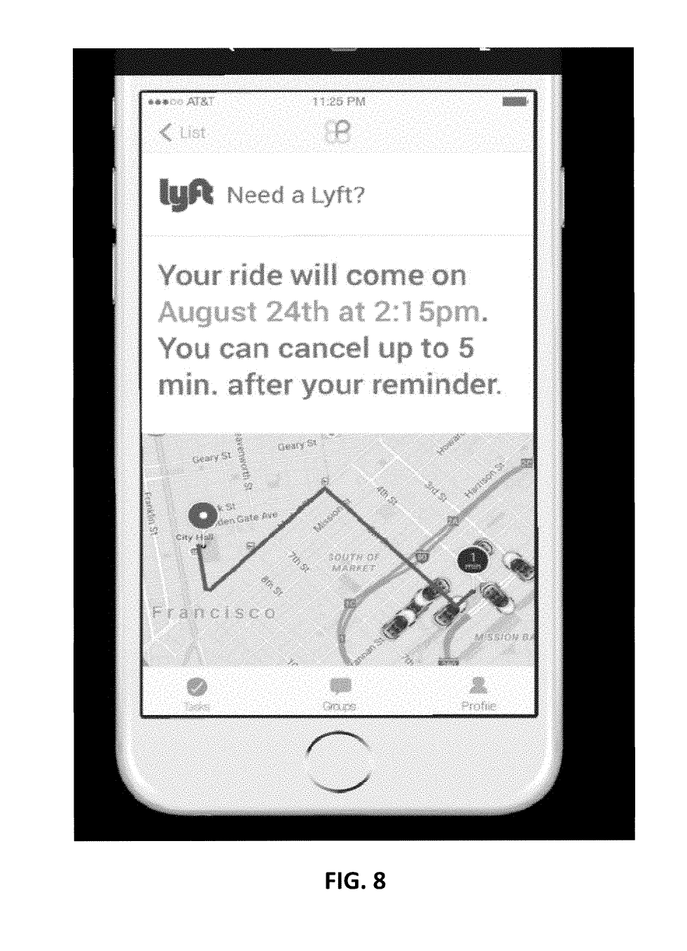

FIG. 8 illustrates an example in which the reminders include a reminder to schedule a ride to a hospital or inpatient facility. In this example, a user may use a ride service (e.g., Lyft™ or other service) to schedule a ride.

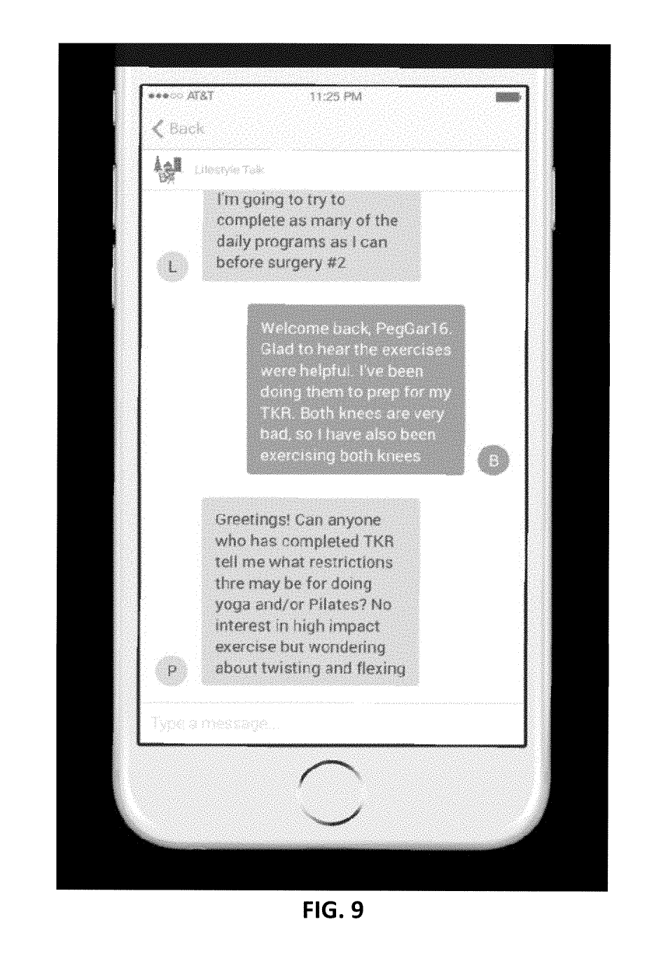

FIG. 9 illustrates an example in which the client messaging supports interactive chatting between patients for them to chat with each other or with an administrator.

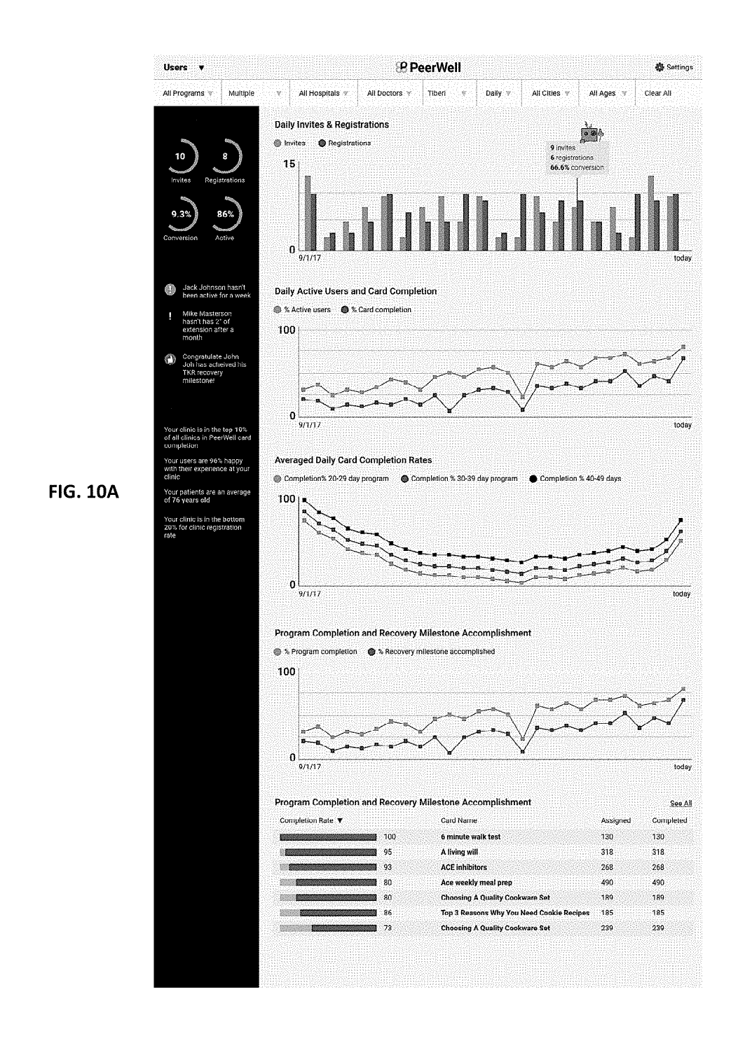

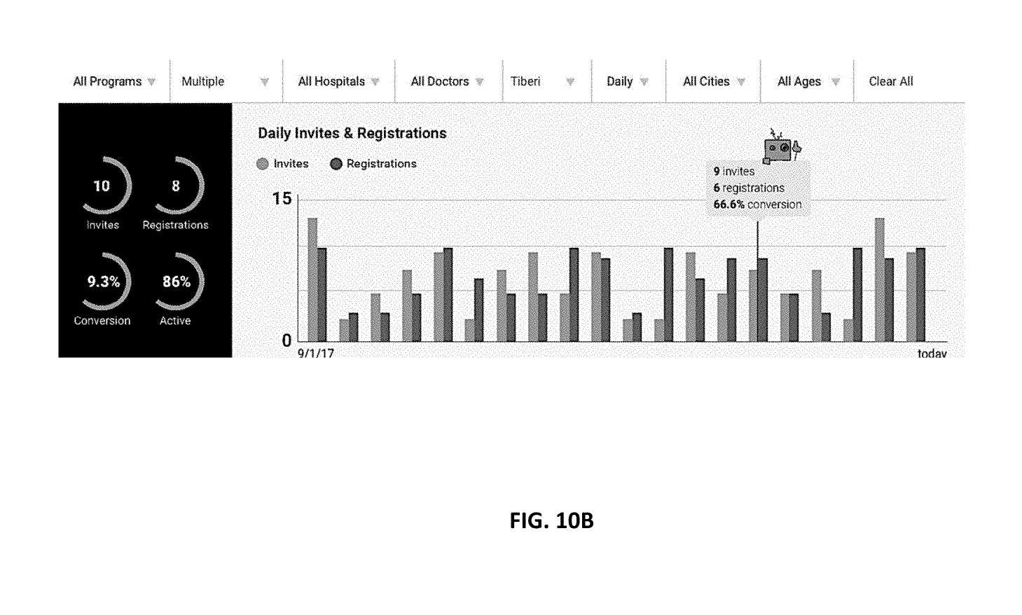

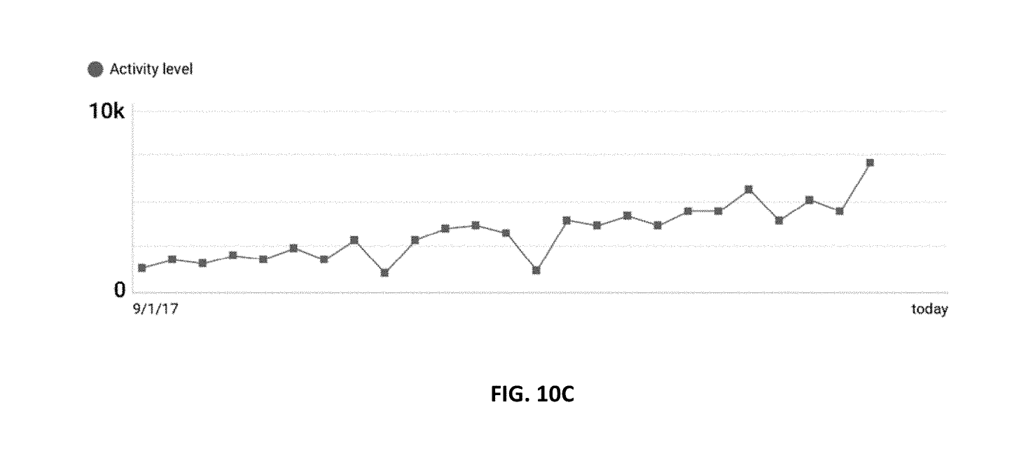

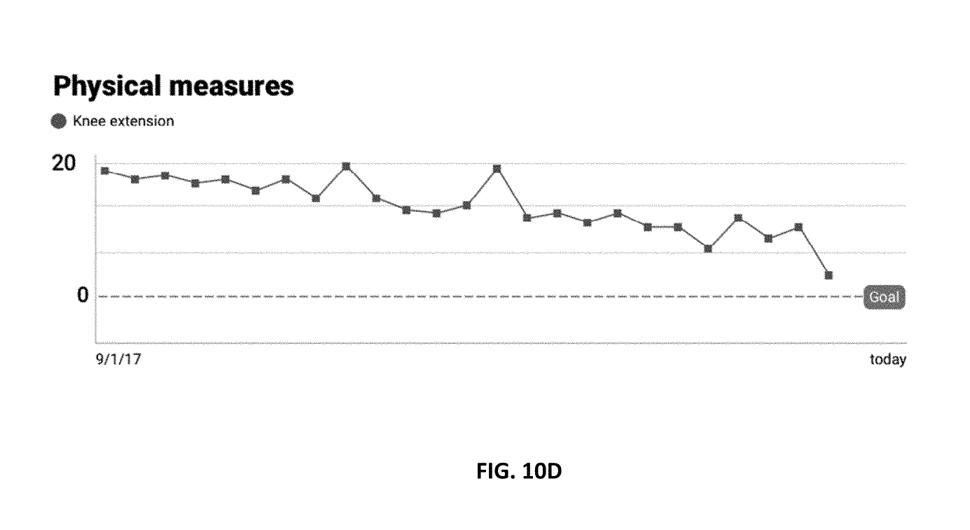

FIG. 10A illustrates an example of an analytical dashboard to monitor the progress of individual patients or groups of patients. This can include statistics on individual patients, groups of patients, and outcomes. It can also display information on program completion rates, program completion and recovery milestone accomplishment data, and data on assignment and completion of individual program module elements. In one embodiment, it also permits a display of data for individual patients showing data on patient education modules they were assigned and have completed. Additional analysis of the data collected for an individual patient may also be displayed. FIG. 10B illustrates an example of a portion of the analytical dashboard showing a high-level overview of daily invites and registrations. FIG. 10C shows a portion of the analytical dashboard showing an activity level of daily active users. FIG. 10D illustrates an example of a portion of the analytical dashboard showing physical measure (e.g., knee extension) for a patient relative to a goal.

Additional Example Methods

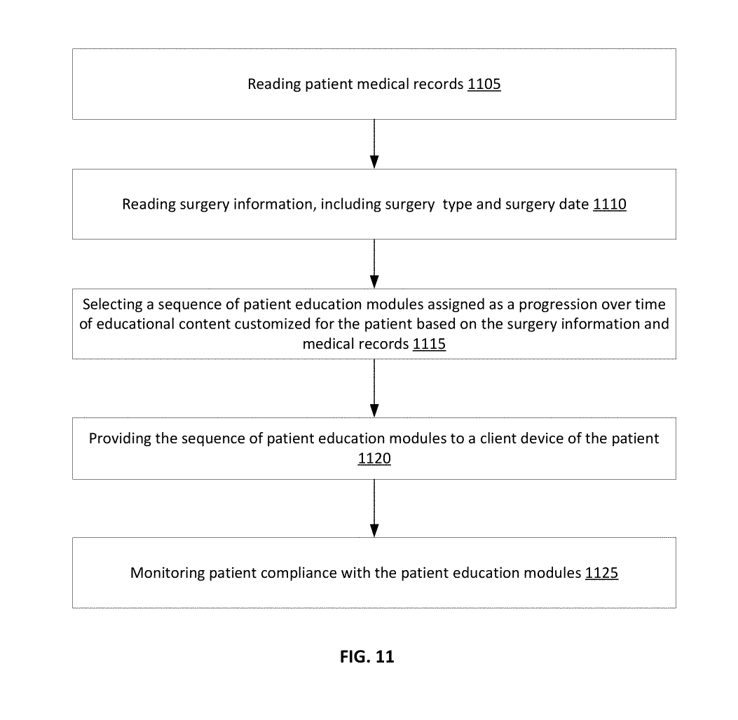

FIG. 11 is a flowchart of a method in accordance with an embodiment. Patient medical records are read in block 1105. This includes reading of surgery information, which may include surgery type and surgery date relative to a current data in block 1110. In block 115, a selection is made of a sequence of patient education modules assigned to the patient as a progression over time customized for the patient based at least in part on their medical records and the surgery information. In block 1120 the sequence is provided to a client device of a patient. For example, the sequence may include a certain number of days (e.g., 30 days, 45 days, 60 days) in which educational content is spread out. In block 1125 the patient's compliance with the patient education module is monitored. This may include any of the previously described scoring or monitoring techniques. Additionally, in some embodiments the sequence of patient education modules may be adjusted based on scores of how well the patient has completed previous patient education modules or how well the patient has performed recommended educational instructions, such as following exercise instructions, or any sensor data received from the patient.

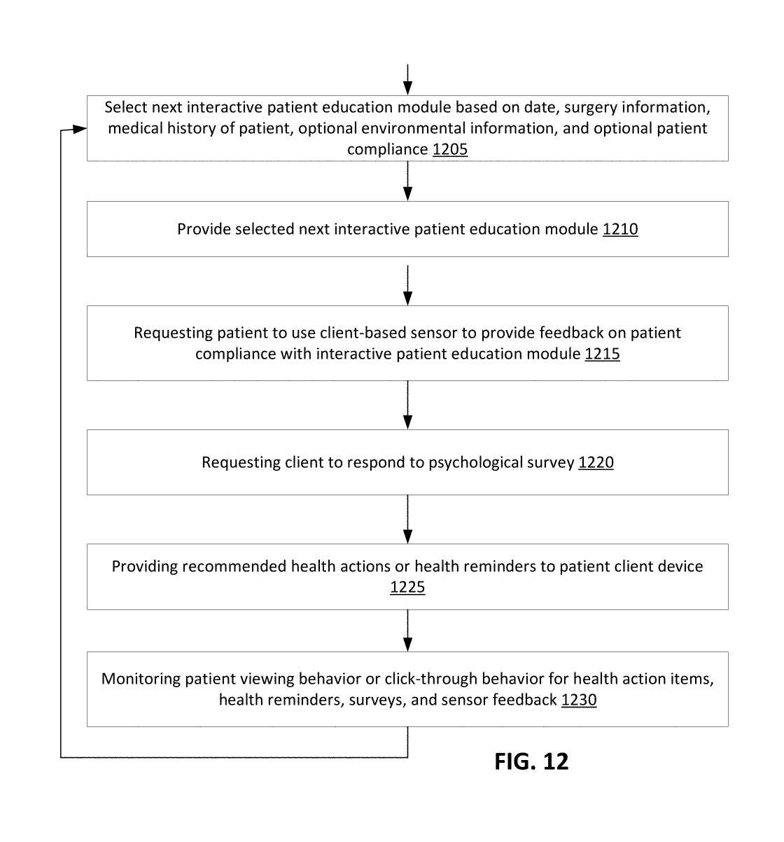

FIG. 12 illustrates an example in which a current patient education module is adapted based on that patient's responses to previous patient education modules. In block 1205, a next patient education module is selected based on different factors and is provided to the patient in block 1210. This education module may include having the patient provide sensor feedback in block 1215, having the patient respond to a psychological survey in block 1220, such as a depression screen or pain management survey, providing recommended health actions or reminders to the patient in block 1225, and monitoring the patient viewing behavior in block 1230. The combined information gathered may then, in turn be used to aid in selecting the next patient education module. For example, if a patient reports a high pain level, the next patient education module may include additional pain management training or surveys. Additionally, the next patient education module could also vary or adapt physical exercises based on a pain survey. More generally, multiple factors may be assessed in a patient education module and used to aid in selecting a next patient education module.

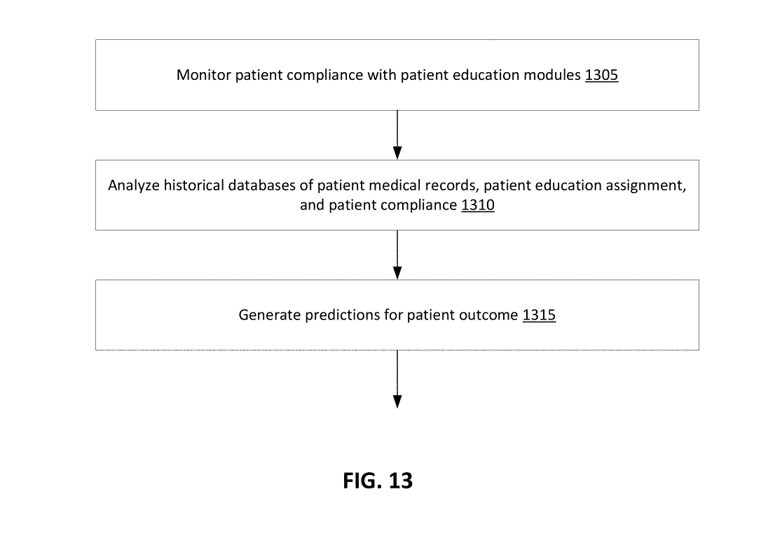

FIG. 13 is a flowchart illustrating a general method of generating predictions based on historical analysis. Patient compliance with patient education modules is monitored in block 1305. In block 1310, the historical database of patient medical records, patient education module assignments and compliance is analyzed. In block 1315, predictions are generated for patient outcomes in a particular case, where the prediction would be of a statistical nature relative such as a greater or less probability of an outcome relative to a typical outcome.

Alternate Embodiments

A system and method providing a series of patient education materials which are responsive to individual patient circumstance and adapted based on new information acquired by the patient or from relevant external sources have been described. In some embodiments, the above-described methods may be implemented in part based on computer program instructions stored in a non-transitory computer readable medium.

In the above description, for purposes of explanation, numerous specific details are set forth in order to provide a thorough understanding of the techniques introduced above. It will be apparent, however, to one skilled in the art that the techniques can be practiced without these specific details. In other instances, structures and devices are shown in block diagram form in order to avoid obscuring the description and for ease of understanding. For example, the techniques are described in one embodiment above primarily with reference to software and particular hardware. However, the present disclosure applies to any type of computing system that can receive data and commands, and present information as part of any peripheral devices providing services.

Reference in the specification to “one embodiment” or “an embodiment” means that a particular feature, structure, or characteristic described in connection with the embodiment is included in at least one embodiment. The appearances of the phrase “in one embodiment” in various places in the specification are not necessarily all referring to the same embodiment.

Some portions of the detailed descriptions described above are presented in terms of algorithms and symbolic representations of operations on data bits within a computer memory. These algorithmic descriptions and representations are, in some circumstances, used by those skilled in the data processing arts to convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing”, “generating”, “computing”, “calculating”, “determining”, “displaying”, or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques also relate to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, and magnetic disks, read-only memories (ROMs), random access memories (RAMs), EPROMs, EEPROMs, magnetic or optical cards, flash memories including USB keys with non-volatile memory or any type of media suitable for storing electronic instructions, each coupled to a computer system bus.

Some embodiments can take the form of an entirely hardware embodiment, an entirely software embodiment or an embodiment containing both hardware and software elements. One embodiment is implemented in software, which includes but is not limited to firmware, resident software, microcode, etc.

Furthermore, some embodiments can take the form of a computer program product accessible from a non-transitory computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

A data processing system suitable for storing and/or executing program code can include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. In addition, the techniques are not described with reference to any particular programming language. It will be appreciated that a variety of programming languages may be used to implement the teachings of the various embodiments as described herein.

The foregoing description of the embodiments has been presented for the purposes of illustration and description. It is not intended to be exhaustive or to limit the specification to the precise form disclosed. Many modifications and variations are possible in light of the above teaching. It is intended that the scope of the embodiments be limited not by this detailed description, but rather by the claims of this application. As will be understood by those familiar with the art, the examples may be embodied in other specific forms without departing from the spirit or essential characteristics thereof. Likewise, the particular naming and division of the modules, routines, features, attributes, methodologies and other aspects are not mandatory or significant, and the mechanisms that implement the description or its features may have different names, divisions and/or formats. Furthermore, the modules, routines, features, attributes, methodologies and other aspects of the specification can be implemented as software, hardware, firmware or any combination of the three. Also, wherever a component, an example of which is a module, of the specification is implemented as software, the component can be implemented as a standalone program, as part of a larger program, as a plurality of separate programs, as a statically or dynamically linked library, as a kernel loadable module, as a device driver, and/or in every and any other way known now or in the future. Additionally, the specification is in no way limited to embodiment in any specific programming language, or for any specific operating system or environment. Accordingly, the disclosure is intended to be illustrative, but not limiting, of the scope of the specification.

What is claimed is:

1. A system for managing patient education pre-surgery or post-surgery, comprising:
a patient education database storing a set of patient education modules;
a patient database storing medical history information for a set of patients;
a network interface to communicate with patient client devices;
a controller to select a sequence of patient education modules, provided via the network interface to the client devices of the set of patients, wherein each patient receives a progression of education content with respect to an upcoming scheduled surgery or a previous surgery with the progression of education content customized for the patient based at least on a type of surgery and the medical history information of the patient with the controller monitoring patient compliance with the patient education modules.

2. The system of claim 1, wherein the controller monitors compliance with at least one of physical exercise instructions, dietary instructions, pain management training, anti-anxiety training, and depression screening.

3. The system of claim 2, wherein the controller customizes the patient education based on feedback from at least one of the monitored patient physical exercise, patient dietary intake, patient pain management training, patient anti-anxiety training, and patient depressions screening.

4. The system of claim 3, wherein the controller stores historical data associating patient medical information, assigned patient education modules for each patient, and the feedback received for each patient in response to assigned patient education modules.

5. The system of claim 2, wherein the controller utilizes sensors of a client device to monitor at least one of physical exercise of a patient, food consumption of a patient, sleep habits of a patient, weight of a patient, breathing of a patient, heart rate of a patient, wound images, and heart rate variability of a patient.

6. The system of claim 1, wherein the controller utilizes sensors of a client device to detect an attribute of a living space or a work space of the patient, determine potential hazards to the patient from the detected attribute, and recommend changes to the living space or the work space.

7. The system of claim 1, wherein the controller adapts the progression of education content in the sequence of patient education modules based on monitoring responses or sensor data submitted from the client device of the patient for one or more patient education modules.

8. The system of claim 1, wherein the system generates a historical database of patient outcomes in response to a particular type of surgery for different sequences of patient education modules, patient compliance in response to the patient education modules assigned to them, and patient medical history information.

9. The system of claim 8, further comprising a patient education engine utilizing the historical database to determine, for each patient, an optimum sequence of patient education modules.

10. The system of claim 8, further comprising an artificial intelligence module trained, based on the historical data, to select the optimum sequence of patient education modules.

11. The system of claim 8, further comprising a prediction engine utilizing the historical database to predict a statistically likely outcome for a patient relative to outcomes of patients in the historical database having a similar operation, similar medical history, and similar compliance behavior with respect to assigned patient education modules.

12. The system of claim 11, further comprising an artificial intelligence module trained from the historical database to identify patients having a higher than average risk for the surgery, a lower than average risk for the surgery, patients that are good candidates for outpatient surgery, and identifying likely rehabilitation outcomes after the surgery.

13. The system of claim 1, wherein the controller selects a sequence of patient education modules including a sequence of educational reminders and notifications for the patient.

14. A method of managing patient education pre-surgery or post-surgery, the method comprising:
selecting, by a controller, an interactive sequence of patient education modules provided to a client device of a patient over a period of time with respect to a scheduled upcoming surgery or a previous surgery with the education content and progression of content customized for the patient based on a type of surgery and medical information of the patient; and
monitoring at least one attribute of patient compliance with each patient education module in the interactive sequence of patient education modules.

15. The method of claim 13, further comprising scoring patient compliance with each patient education module by at least one scoring technique.

16. The method of claim 13, further comprising adapting the sequence of patient education modules based on patient responses to the patient education modules.

17. The method of claim 13, wherein at least one of the patient education modules includes a pain survey or pain management training.

18. The method of claim 13, wherein at least one of the patient education modules is a depression survey.

19. The method of claim 13, wherein at least one of the patient education modules is a dietary module to improve a diet of a patient.

20. The method of claim 13, wherein at least one of the patient education modules is an exercise module.

21. The method of claim 13, wherein at least one of the patient education modules is an interactive module that includes utilizing at least one sensor of a client device of the patient to monitor a patient health attribute associated with a patient education module.

22. The method of claim 20, wherein the at least one sensor comprises at least one of a microphone, a camera, a gyroscope, and an accelerometer of a patient client device.

23. The method of claim 20, wherein the at least one sensor comprises a sensor of a smart fitness watch, a smart scale, a smart appliance, a sleep monitor, or a heart monitor.

24. The method of claim 13, further comprising utilizing at least one dynamic external data source to select content for a patient education module.

25. The method of claim 13, further comprising performing a prediction analysis of an outcome of the surgery based on compliance of a patient with the sequence of patient education modules and the scoring results.

26. The method of claim 24, wherein the prediction analysis comprises predicting a relative risk of an inpatient outcome to an outpatient outcome.

27. The method of claim 13, wherein at least one patient education module is directed to preparing a residence of a patient.

28. The method of claim 13, further comprises generating a sequence of health reminders and notifications for the patient.

29. A method of providing patient education pre-surgery and post-surgery, the method comprising:
selecting, by a controller, a first interactive sequence of patient education modules provided to a client device of a patient over a period of time prior to a scheduled surgery with the education content and progression of content selected based on a likelihood of achieving a desired prehabilitation outcome;
monitoring at least one attribute of patient compliance with each patient education module in the first sequence of patient education modules; and
selecting, by the controller, a second interactive sequence of patient education modules provided to a client device of patient over a period of time after the scheduled surgery with the education content and progression of content selected based on a likelihood of achieving a desired outcome for rehabilitation; and
monitoring at least one attribute of patient compliance with each patient education module.
